(19) United States
(12) Patent Application Publication
(10) Pub. No.: US 2022/0336064 A1
(43) Pub. Date: Oct. 20, 2022

(54) MEDICAL INFORMATION PROCESSING APPARATUS

(71) Applicant: CANON MEDICAL SYSTEMS CORPORATION, Otawara-shi (JP)

(72) Inventors: Masashi YOSHIDA, Nasushiobara (JP); Yasunori OHSHIMA, Yaita (JP); Daizo OIKAWA, Otawara (JP); Hideo SAITO, Nasushiobara (JP)

(73) Assignee: CANON MEDICAL SYSTEMS CORPORATION, Otawara-shi (JP)

(21) Appl. No.: 17/659,213

(22) Filed: Apr. 14, 2022

(30) Foreign Application Priority Data
Apr. 19, 2021 (JP) 2021-070587

(51) Int. Cl. G16H 10/60; G16H 40/20; G16H 70/20

(52) U.S. Cl. CPC G16H 10/60; G16H 40/20; G16H 70/20

(57) ABSTRACT

A medical information processing apparatus comprises processing circuitry. The processing circuitry collects patient information and medical information related to a medical record including a plurality of medical actions. The processing circuitry causes a display to display the medical record by displaying the plurality of medical actions, the collected patient information, and the collected medical information in time series and displaying an option selected in the medical record by a doctor in association with any reason for the selection of the option. The processing circuitry causes the display device to display the option selected by the doctor and any option not selected by the doctor in a distinguishable manner.

### CROSS-REFERENCE TO RELATED APPLICATIONS

This application is based upon and claims the benefit of priority from Japanese Patent Application No. 2021-070587, filed on Apr. 19, 2021; the entire contents of which are incorporated herein by reference.

### FIELD

Embodiments described herein relate generally to a medical information processing apparatus.

### BACKGROUND

Conventionally, at a medical facility, a medical action such as a medical treatment has been performed by a doctor based on information such as an examination result related to a patient. Recently, a support technology has been disclosed such as a clinical decision support system (CDSS) configured to infer the disease state, conversion risk, and the like of a target patient as a medical care target by using a model obtained by analyzing past medical care information of the patient by technology such as machine learning.

However, in an ex-post facto revision of a medical action, detailed verification has been difficult in some cases due to insufficient recording of information at a timing when the medical action is performed and information on the basis of determination by a doctor. Furthermore, ex-post facto verification has been burdensome in some cases when various records are available but not associated with individual medical actions.

### BRIEF DESCRIPTION OF THE DRAWINGS

FIG. 1 is a diagram illustrating an exemplary configuration of a medical information processing system according to a first embodiment;

FIG. 2 is a diagram illustrating an exemplary configuration of a clinical decision support device according to the first embodiment;

FIG. 3 is a diagram illustrating an exemplary workflow in progress according to the first embodiment;

FIG. 4 is a diagram illustrating an exemplary selection screen for a medical action candidate according to the first embodiment;

FIG. 5 is a diagram illustrating an exemplary selection screen for a selection reason candidate according to the first embodiment;

FIG. 6 is a diagram illustrating an exemplary browsing screen for an executed workflow according to the first embodiment;

FIG. 7 is a diagram illustrating an exemplary continuation of the workflow browsing screen in FIG. 6;

FIG. 8 is a flowchart illustrating an exemplary process of processing of supporting clinical decision according to the first embodiment;

FIG. 9 is a flowchart illustrating an exemplary process of processing of supporting ex-post facto analysis or verification related to a medical action according to the first embodiment;

FIG. 10 is a diagram illustrating an exemplary configuration of a clinical decision support device according to a second embodiment;

FIG. 11 is a diagram illustrating an exemplary browsing screen for an executed workflow according to the second embodiment;

FIG. 12 is a diagram illustrating an exemplary search condition table according to a third embodiment;

FIG. 13 is a diagram illustrating an exemplary browsing screen for an executed workflow according to the third embodiment;

FIG. 14 is a diagram illustrating an exemplary emphatic display related to a selected medical treatment strategy according to the third embodiment;

FIG. 15 is a diagram illustrating an exemplary emphatic display related to a medical treatment strategy different from that in FIG. 14;

FIG. 16 is a diagram illustrating an exemplary browsing screen for an executed workflow according to a fourth embodiment;

FIG. 17 is a diagram illustrating an exemplary information obtainable timing related to a past medical action according to the fourth embodiment;

FIG. 18 is a diagram illustrating an exemplary selection screen for a medical action candidate according to a first modification;

FIG. 19 is a diagram illustrating an exemplary selection screen for a selection reason candidate according to the first modification; and

FIG. 20 is a diagram illustrating an exemplary browsing screen for an executed workflow according to the first modification.

### DETAILED DESCRIPTION

A medical information processing apparatus comprises processing circuitry. The processing circuitry collects patient information and medical information related to a medical record including a plurality of medical actions. The processing circuitry causes a display device to display the medical record by displaying the plurality of medical actions, the collected patient information, and the collected medical information in time series and displaying an option selected in the medical record by a doctor in association with any reason for the selection of the option. The processing circuitry causes the display to display the option selected by the doctor and any option not selected by the doctor in a distinguishable manner.

Embodiments of a medical information processing apparatus will be described below in detail with reference to the accompanying drawings.

### First Embodiment

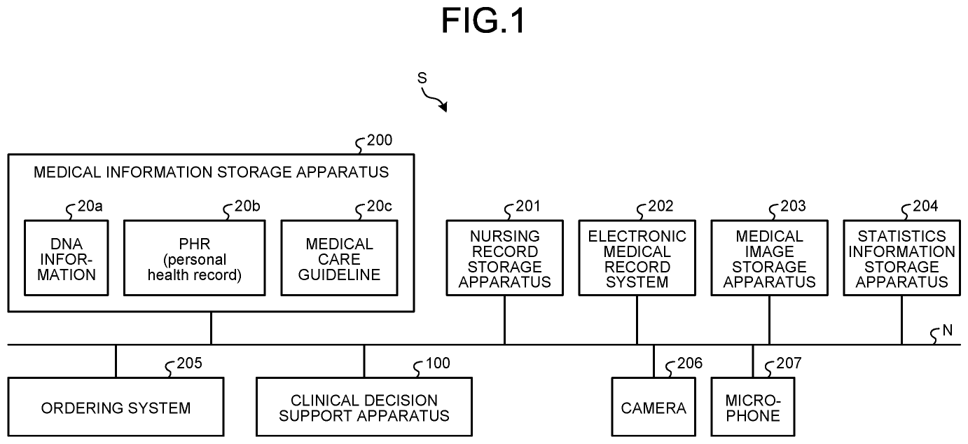

FIG. 1 is a diagram illustrating an exemplary configuration of a medical information processing system S according to a first embodiment. As illustrated in FIG. 1, the medical information processing system S includes a clinical decision support apparatus 100, a medical information storage apparatus 200, a nursing record storage apparatus 201, an electronic medical record system 202, a medical image storage apparatus 203, a statistics information storage apparatus 204, an ordering system 205, a camera 206, and a microphone 207. These components are connected to each other to perform communication therebetween through a network N such as an in-facility local area network (LAN) or the Internet.

The clinical decision support apparatus 100 of the present embodiment supports a clinical decision related to a medical action by a doctor. The clinical decision support apparatus 100 records a selection result of the medical action by the doctor and patient information and medical information related to the medical action. The clinical decision support apparatus 100 is configured as, for example, a server device or a personal computer (PC). Functions of the clinical decision support apparatus 100 will be described later in detail. A doctor is an example of a user of the clinical decision support apparatus 100 in the present embodiment. The clinical decision support apparatus 100 is an example of a medical information processing apparatus of the present embodiment.

A medical action in the present embodiment includes general medical actions such as a medical examination, an examination, and a medical treatment.

Patient information in the present embodiment is information related to a medical action target patient and includes, for example, property information, an electronic medical record, a nursing record, an examination result, a medical image, deoxyribonucleic acid (DNA) information, or a personal health record (PHR) of the patient. The property information of a patient is information of the sex, age, height, weight, and the like of the patient.

Medical information in the present embodiment includes, for example, medical statistics information and a medical care guideline.

The medical information processing system S is provided at a medical institution such as a hospital.

The medical information storage apparatus 200 stores DNA information 20a, a PHR 20b, a medical care guideline 20c, and the like of a patient. The DNA information 20a, the PHR 20b, and the medical care guideline 20c may be stored in devices different from each other.

The nursing record storage apparatus 201 is a device configured to store a nursing record related to nursing of a patient. The electronic medical record system 202 is a device configured to store an electronic medical record.

The medical image storage apparatus 203 is, for example, a server device of a picture archiving and communication system (PACS) and stores medical image data in a format compliant to digital imaging and communications in medicine (DICOM). A medical image is, for example, computed tomography (CT) image data, magnetic resonance image data, or ultrasonic wave diagnosis image data but not limited to them.

The statistics information storage apparatus 204 is a device configured to store a database that stores medical statistics information.

The ordering system 205 is a device configured to issue an examination order to a non-illustrated radiology information system (RIS), a prescription order to a non-illustrated pharmacy department system, or the like based on an operation by a medical care doctor or the like.

The medical information storage apparatus 200, the nursing record storage apparatus 201, the electronic medical record system 202, the medical image storage apparatus 203, the statistics information storage apparatus 204, and the ordering system 205 as hardware are each a computer such as a server device. Some of these devices may be integrated with each other. For example, functions of a plurality of devices illustrated in FIG. 1 may be integrated with each other as a hospital information system (HIS).

The camera 206 is provided at, for example, an examination room and captures an image of an action of an examination or a medical treatment on a patient. The microphone 207 is provided at, for example, an examination room and collects voice of a medical professional such as a doctor. The camera 206 and the microphone 207 may be provided to the clinical decision support apparatus 100. The camera 206 and the microphone 207 may be collectively referred to as a sensor.

The configuration illustrated in FIG. 1 is exemplary, and the medical information processing system S may additionally include any other component and does not necessarily need to include part of the configuration illustrated in FIG. 1. For example, the medical information processing system S may include a RIS or a pharmacy department system.

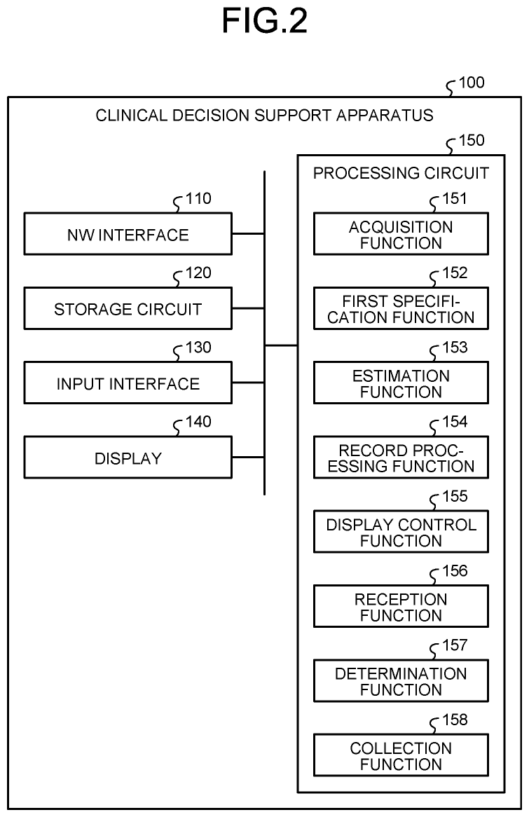

The following describes the functions of the clinical decision support apparatus 100. FIG. 2 is a diagram illustrating an exemplary configuration of the clinical decision support apparatus 100 according to the first embodiment.

The clinical decision support apparatus 100 includes a network (NW) interface circuit 110, a memory 120, an input interface circuit 130, a display device 140, and a processing circuit 150.

The NW interface circuit 110 is connected to the processing circuit 150 and controls transmission of various kinds of data and communication between the clinical decision support apparatus 100 and any other information processing apparatus. The NW interface circuit 110 is implemented with a network card, a network adapter, a network interface controller (NIC), or the like.

The memory 120 stores in advance various kinds of information used by the processing circuit 150. For example, the memory 120 of the present embodiment stores a workflow of a medical task including a plurality of medical actions, patient information or medical information associated with each medical action included in the workflow, a reason for a determination by the doctor, and the like.

The memory 120 also stores various computer programs. The memory 120 is implemented with, for example, a semiconductor memory element such as a random access memory (RAM) or a flash memory, an optical disk, a hard disk drive (HDD), or a solid state drive (SSD). The memory 120 may be located in the clinical decision support apparatus 100 or in, for example, an external storage device connected to the clinical decision support apparatus 100 through the network N.

The input interface circuit 130 is implemented with a truck ball, a switch button, a mouse, a keyboard, a touch pad through which an input operation is performed with touch on an operation surface, a touch screen as an integration of a display screen and a touch pad, a non-contact input circuit using an optical sensor, a voice input circuit, or the like. The input interface circuit 130 is connected to the processing circuit 150, converts an input operation received from an operator into an electric signal, and outputs the electric signal to the processing circuit 150. The input interface circuit 130 in the present specification is not limited to a physical operation member such as a mouse or a keyboard. Examples of the input interface circuit 130 include an electric signal processing circuit configured to receive an electric signal corresponding to an input operation from an external input instrument provided separately from the clinical decision support apparatus 100 and output the electric signal to the processing circuit 150.

The display device 140 is a dot-matrix electronic display such as a liquid crystal display or an organic electro-luminescence (organic EL or OEL) display. The input interface circuit 130 may be integrated with the display device 140. For example, the input interface circuit 130 and the display device 140 may be implemented with a touch panel. The display device 140 is an example of a display device in the present embodiment. The display device 140 may be separated from the clinical decision support apparatus 100 and connected to the clinical decision support apparatus 100 through, for example, the network N.

The processing circuit 150 is a processor configured to read computer programs from the memory 120 and execute the computer programs, thereby achieving a function corresponding to each computer program. The processing circuit 150 of the present embodiment includes an acquisition function 151, a first specification function 152, an estimation function 153, a record processing function 154, a display control function 155, a reception function 156, a determination function 157, and a collection function 158.

For example, processing functions of the acquisition function 151, the first specification function 152, the estimation function 153, the record processing function 154, the display control function 155, the reception function 156, the determination function 157, and the collection function 158 as constituent components of the processing circuit 150 are stored in the memory 120 in the form of computer-executable program. The processing circuit 150 is a processor. For example, the processing circuit 150 reads computer programs from the memory 120 and executes the computer programs, thereby achieving a function corresponding to each computer program. In other words, the processing circuit 150 having read a computer program has the corresponding function illustrated in the processing circuit 150 in FIG. 2. In FIG. 2, the processing functions performed at the acquisition function 151, the first specification function 152, the estimation function 153, the record processing function 154, the display control function 155, the reception function 156, the determination function 157, and the collection function 158 are implemented by a single processor, but the processing circuit 150 may be configured by combining a plurality of independent processors and each processor executes a computer program to implement the corresponding function. In FIG. 2, each computer program corresponding to a processing function is stored in the single memory 120, but a plurality of storage circuits may be disposed in a distributed manner and each computer program may be read from the corresponding storage circuit by the processing circuit 150.

Although the above description is made on an example in which a “processor” reads each computer program corresponding to a function from a storage circuit and executes the computer program, the embodiment is not limited thereto. The term “processor” means a circuit such as a central processing unit (CPU), a graphics processing unit (GPU), an application specific integrated circuit (ASIC), or a programmable logic device (for example, a simple programmable logic device (SPLD), a complex programmable logic device (CPLD), or a field programmable gate array (FPGA)). When the processor is, for example, a CPU, the processor implements a function by reading and executing a computer program stored in the memory 120. When the processor is an ASIC, a computer program is not stored in the memory 120 but the corresponding function is directly incorporated as a logic circuit in a circuit of the processor. Each processor of the present embodiment is not limited to a configuration in which each processor is configured as a single circuit, but one processor may be configured by combining a plurality of independent circuits to implement any function. Alternatively, the plurality of constituent components in FIG. 2 may be integrated into one processor to implement their functions.

The clinical decision support apparatus 100 may include various medical applications such as an image diagnosis application in addition to the functions illustrated in FIG. 2. A computer program of the medical application is, for example, stored in the memory 120 and executed by the processing circuit 150.

The clinical decision support apparatus 100 of the present embodiment has a function to support a clinical decision when a doctor executes a medical action and recording of information related to the clinical decision, and a function to support ex-post facto analysis or verification of an already executed medical action. A doctor who executes a medical action and a doctor who executes ex-post facto analysis or verification of the already executed medical action do not necessarily need to the same person. The following describes individual functions of the clinical decision support apparatus 100 in detail.

The acquisition function 151 acquires patient information and medical information from various devices included in the medical information processing system S. The acquisition function 151 also acquires, from the camera 206, a video obtained by capturing an examination room or the like. The acquisition function 151 also acquires collected voice from the microphone 207.

When a medical action is performed by a doctor or the like, the first specification function 152 specifies a workflow currently in progress among a plurality of workflows stored in the memory 120. The first specification function 152 searches the memory 120 for a corresponding workflow based on, for example, patient identification information input by the doctor.

In a workflow, a plurality of medical actions are associated in time series. A workflow may include not only an already executed medical action but also a medical action yet to be executed in a plan. For example, a workflow is constituted by a plurality of medical actions associated in time series based on a medical treatment plan generated based on a clinical path or the like. A medical treatment plan on which a workflow is based, for example, may be generated by the estimation function 153 to be described later or may be generated outside the clinical decision support apparatus 100. A medical treatment plan is stored in, for example, the memory 120.

A workflow is an example of a medical record in the present embodiment. In the present embodiment, a medical action included in a workflow is also referred to as a step.

In addition, the first specification function 152 specifies an execution target step among a plurality of medical actions included in a workflow 7a.

Alternatively, the first specification function 152 may specify a workflow currently in progress and an execution target step in the workflow based on the video acquired from the camera 206 or the voice acquired from the microphone 207. Alternatively, the first specification function 152 may specify a workflow currently in progress and an execution target step in the workflow based on the patient information and the medical information acquired from various devices included in the medical information processing system S. Alternatively, the first specification function 152 may specify an execution target step based on an operation by the user on a medical application that is executed at the clinical decision support apparatus 100 or an analysis result of the medical application. Alternatively, the first specification function 152 may specify an execution target step based on a combination result of these pieces of information.

For example, when having detected a catheter from the video captured by the camera 206 in a case in which the workflow 7a includes a step related to a surgical operation using a catheter, the first specification function 152 determines that a transition has been made from a surgical operation preparation step to a catheter insertion step in the workflow 7a. In this case, the first specification function 152 specifies the catheter insertion step as a current execution target step.

For example, when CT examination image data is stored in the medical image storage apparatus 203, the first specification function 152 may determine that an image capturing step by an X-ray CT device has ended and an image analysis step has started. For example, when a report application for referral of a radiogram interpretation report is activated among medical applications, the first specification function 152 may determine that a radiogram interpretation step has ended and a report step has started.

The user may select an execution target step. For example, a step planned next to a latest step among executed steps becomes an execution target when the user presses down a non-illustrated button such as “to the next step” displayed on the display device 140. For example, the user may select, as an execution target, a step registered in the workflow but yet to be executed.

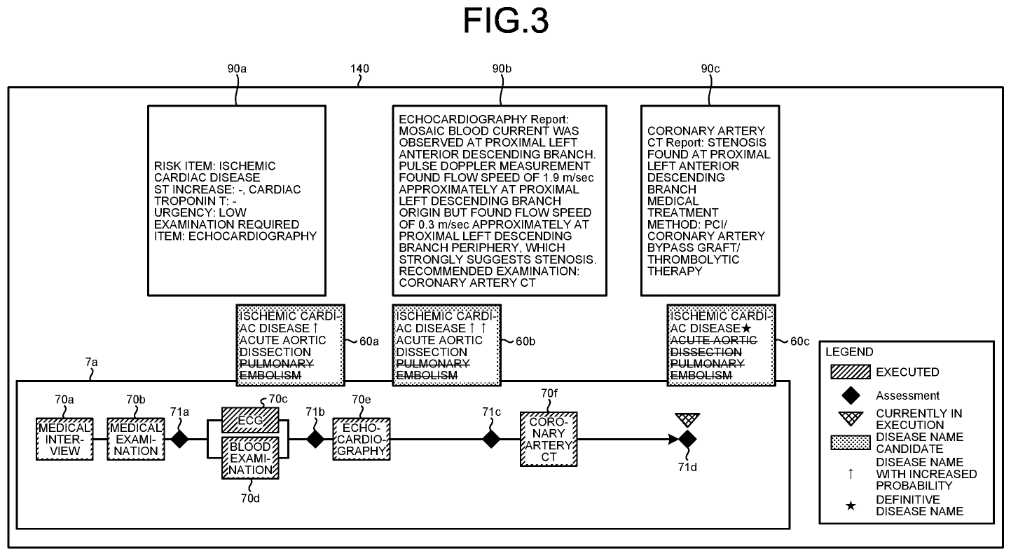

FIG. 3 is a diagram illustrating an exemplary workflow 7a in progress according to the first embodiment. The workflow 7a illustrated in FIG. 3 is displayed on the display device 140 by the display control function 155 to be described later.

In the present embodiment, a plurality of medical actions include nodes 70a to 70f corresponding to medical actions involving tasks such as a medical interview, a medical examination, an examination, and a medical treatment action, and also include bifurcation points 71a to 71d corresponding to an assessment for discussing a next task. Hereinafter, the individual nodes 70a to 70f are simply referred to as nodes 70 when not distinguished. The individual bifurcation points 71a to 71d are simply referred to as bifurcation points 71 when not distinguished. In FIG. 3, a medical action and an assessment are distinguished for sake of convenience, but “assessment” may be included in the definition of “medical action”. The nodes 70 and the bifurcation points 71 are each an example of a step included in the workflow 7a.

In the example illustrated in FIG. 3, the bifurcation point 71d corresponding to an assessment after the node 70f of coronary artery CT is a step in execution among a plurality of steps included in the workflow 7a. The workflow 7a illustrated in FIG. 3 is exemplary and is not restrictive.

Up to a definitive diagnosis, disease name candidates are narrowed down at stages based on information obtained through a medical interview and an examination as illustrated in FIG. 3. Thus, for example, “determination of questions in a medical interview” and “determination of an examination to be performed” are main decisions by a doctor before the definitive diagnosis.

As illustrated in FIG. 2, when a medical action is to be performed by a doctor or the like, the estimation function 153 estimates any candidate for a medical action to be executed next based on patient information and medical information related to a medical action currently in progress.

In addition, the estimation function 153 estimates the name of any disease having a high probability to be suffered by a patient based on a result of any examination performed up to the bifurcation point 71d. When a plurality of examinations are performed, the estimation function 153 estimates an excluded disease name, the name of a disease having an increased probability, or the name of a disease having a high risk, based on a result of each examination. The name of an estimated disease varies in accordance with results of a medical interview and an examination. Typically, processing of collecting information through a medical interview or an examination and processing of estimation based on the collected information are repeated until a disease name is narrowed down. For example, in the example illustrated in FIG. 3, the probability of “lung thrombus” is excluded among the disease name candidates in a disease name candidate display field 60a at an assessment corresponding to the bifurcation point 71b after an electrocardiogram (ECG) examination at the node 70c and a blood examination at the node 70d are executed. The probability of “ischemic cardiac disease” increases among the disease name candidates.

At the bifurcation point 71c, the probability of “ischemic cardiac disease” further increases among the disease name candidates. Then, at the bifurcation point 71d, the estimation function 153 estimates that the probability of any disease name other than “ischemic cardiac disease” is excluded based on an examination result. At the bifurcation point 71d, a disease name diagnosis step ends when a doctor has approved “ischemic cardiac disease” as a definitive disease name. A definitive disease name is also referred to as a diagnosis name. The estimation function 153 estimates any candidate for a medical action to be executed next based on a result of any examination up to a disease name diagnosis and the definitive disease name.

The estimation function 153 may be an application of a clinical decision support system (CDSS) employing a well-known technology. Alternatively, the estimation function 153 may be a learning-completed model having learned patient information, medical information, and diagnosis results in the past. The learning-completed model is generated by deep learning or other machine learning, or the like, but may be generated by employing any other well-known technology. A clinical decision support system provided outside the clinical decision support apparatus 100 may be used in place of the estimation function 153. A clinical decision support system may be included in the medical information processing system S.

When having estimated a plurality of medical action candidates, the estimation function 153 determines a medical action candidate to be recommended most among the plurality of medical action candidates together with a recommendation reason.

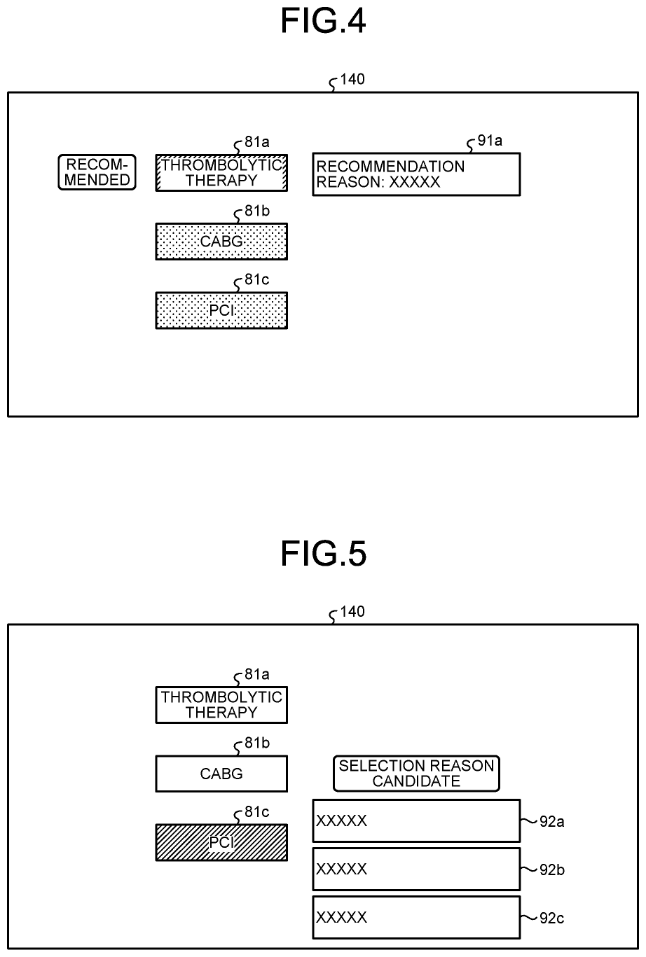

FIG. 4 is a diagram illustrating an exemplary selection screen for a medical action candidate according to the first embodiment; The selection screen illustrated in FIG. 4 is displayed on the display device 140 by the display control function 155 to be described later. In the example illustrated in FIG. 4, the three selection buttons of a selection button 81a indicating “thrombolytic therapy”, a selection button 81b indicating “coronary artery bypass graft (CABG)”, and a selection button 81c indicating “percutaneous coronary intervention (PCI)” are displayed as options.

On the selection screen illustrated in FIG. 4, thrombolytic therapy is displayed as a recommended option. In this case, a recommendation reason determined by the estimation function 153 may be displayed near the selection button 81a indicating “thrombolytic therapy”.

The display aspect of any available recommendation in FIG. 4 is exemplary and not restrictive. The estimation function 153 may generate not only a recommendation reason for an option recommended most but also a recommendation reason for any other option. When either selection button is pressed down by the user, an order for an examination corresponding to the option may be transmitted to the ordering system 205 through the NW interface circuit 110 and the network N.

When a medical action candidate selected by a doctor is different from a medical action candidate recommended most by the estimation function 153, the estimation function 153 estimates any reason candidate for the selection by the doctor. Whether the medical action candidate selected by the doctor is different from the medical action candidate recommended most by the estimation function 153 is determined by the determination function 157 to be described later.

FIG. 5 is a diagram illustrating an exemplary selection screen for a selection reason candidate according to the first embodiment; The selection screen illustrated in FIG. 5 is displayed on the display device 140 by the display control function 155 to be described later.

FIG. 5 exemplarily illustrates a case in which not the option “thrombolytic therapy” recommended most by the estimation function 153 but the option “PCI” is selected by a doctor. In this case, the estimation function 153 estimates, based on patient information and medical information, any reason candidate for the selection of the option by the doctor. For example, the estimation function 153 estimates, as selection reason candidates 92a to 92c, patient information of a target patient of a medical care and medical information related to the option “PCI” selected by the doctor.

The selection reason candidates 92a to 92c include patient preference, allergy existence, pre-existing disease, and past medical history, a simulation result of a result obtained if a medical action corresponding to each option is performed, and the like, which are written in an electronic medical record as an example of the patient information. The preference of the patient is information such as information of whether complete cure of an illness through a medical treatment or beautification of a scar or the like is prioritized. The simulation result of a result obtained if a medical action corresponding to each option is performed may be obtained by the estimation function 153 by using a learning-completed model or the like, or the simulation result obtained by another medical application may be used. The estimation function 153 may include, in the selection reason candidates 92a to 92c, not only an option selected by the doctor but also advantage and disadvantage of each estimated option.

Although the three selection reason candidates 92a to 92c are displayed on the display device 140 in FIG. 5, the number of selection reason candidates is not particularly limited. Hereinafter, the individual selection reason candidates 92a to 92c are simply referred to as selection reason candidates 92 when not particularly distinguished. In addition to the reason candidates estimated by the estimation function 153, an input field in which a reason can be freely input by a doctor may be provided on the display device 140. The number of selection reason candidates that can be selected by a doctor among the selection reason candidates 92 is not particularly limited.

As illustrated in FIG. 2, when a doctor adopts an option different from an option recommended by a clinical decision support system in a situation in which a medical action is executed by the doctor, the record processing function 154 stores the option adopted by the doctor in association with a reason for the selection in the memory 120.

In the present embodiment, when a medical action selected by a doctor is different from a medical action recommended most by the estimation function 153, the record processing function 154 stores, in association with the medical action selected by the doctor in the memory 120, a reason selected by the doctor from among reason candidates estimated by the estimation function 153 for the option selection by the doctor. In the memory 120, a data database in which each medical action selected by a doctor is associated with its selection reason is referred to as a reason record database.

In the example illustrated in FIG. 5, when two of the selection reason candidate 92a and the selection reason candidate 92b are selected by a doctor from among the selection reason candidates 92a to 92c, the record processing function 154 stores the contents of the selection reason candidate 92a and the selection reason candidate 92b in association with a medical action “PCI” in the reason record database of the memory 120.

When a medical action candidate selected by a doctor matches a medical action candidate recommended most by the estimation function 153, the record processing function 154 stores the corresponding recommendation reason generated by the estimation function 153 in association with the medical action in the reason record database.

As illustrated in FIG. 2, the display control function 155 causes the display device 140 to display the workflow 7a and patient information and medical information related to the workflow 7a in a situation in which a medical action is executed by a doctor or a situation in which ex-post facto analysis or verification is performed for an already executed medical action.

More specifically, in a situation in which a medical action is executed by a doctor, the display control function 155 causes the display device 140 to display the workflow 7a specified as an execution target by the first specification function 152 as illustrated in FIG. 3.

In addition, the display control function 155 causes the display device 140 to display information display fields 90a to 90c in which patient information and medical information corresponding to each step included in the workflow 7a are displayed as illustrated in FIG. 3. The information display fields 90a to 90c are displayed at, for example, positions near the respective nodes 70 of the medical actions corresponding to the information display fields 90a to 90c. Specifically, the information display field 90b in which a report result of echocardiography executed at the node 70e is displayed is displayed near the node 70e. Hereinafter, the individual information display fields 90a to 90c are simply referred to as information display fields 90 when not particularly distinguished. Patient information and medical information displayed in the information display fields 90 are also referred to as consult data.

In addition, the display control function 155 causes the display device 140 to display disease name candidate display fields 60a to 60c in which any disease name candidate estimated by the estimation function 153 at each bifurcation point 71 is displayed as illustrated in FIG. 3. Hereinafter, the individual disease name candidate display fields 60a to 60c are simply referred to as disease name candidate display fields 60 when not particularly distinguished. The display control function 155 causes the display device 140 to display any disease name candidate excluded by the estimation function 153 in a display aspect different from that of the other disease name candidates. For example, the display control function 155 causes the display device 140 to display a strike-out line on any excluded disease name candidate. In addition, the display control function 155 adds a symbol indicating probability increase to any disease name candidate estimated to have an increased probability by the estimation function 153. In the example illustrated in FIG. 3, the display control function 155 causes the display device 140 to display upward arrows in a number in accordance with the degree of probability increase near any disease name candidate estimated to have an increased probability by the estimation function 153.

In addition, the display control function 155 adds a symbol to a disease name determined as a definitive disease name by a doctor as illustrated in FIG. 3. In the example illustrated in FIG. 3, the display control function 155 causes the display device 140 to display a star symbol near “ischemic cardiac disease” as a definitive disease name. The display illustrated in FIG. 3 is exemplary and not restrictive.

In a situation in which a medical action is executed by a doctor, the display control function 155 causes the display device 140 to display, as an option on the display device 140, any medical action candidate estimated by the estimation function 153 as illustrated in FIG. 4.

When a medical action candidate selected by the doctor is different from a medical action candidate recommended most by the estimation function 153, the display control function 155 causes the display device 140 to display any selection reason candidate estimated by the estimation function 153 as illustrated in FIG. 5.

In a situation in which ex-post facto analysis or verification is performed for an already executed medical action, when displaying the workflow 7a on the display device 140, the display control function 155 causes the display device 140 to display a plurality of medical actions included in the workflow 7a, patient information, and medical information in time series and causes the display device 140 to display an option selected by a doctor in the workflow 7a in association with any reason for the selection of the option.

The display control function 155 causes the display device 140 to display, in a distinguishable manner, the option selected by the doctor and any other option not selected by the doctor in the workflow 7a.

Patient information and medical information displayed together with the workflow 7a by the display control function 155 are patient information and medical information associated with a medical action as a step included in the workflow 7a. The patient information and the medical information are collected by the collection function 158 to be described later in a situation in which ex-post facto analysis or verification is performed for an already executed medical action.

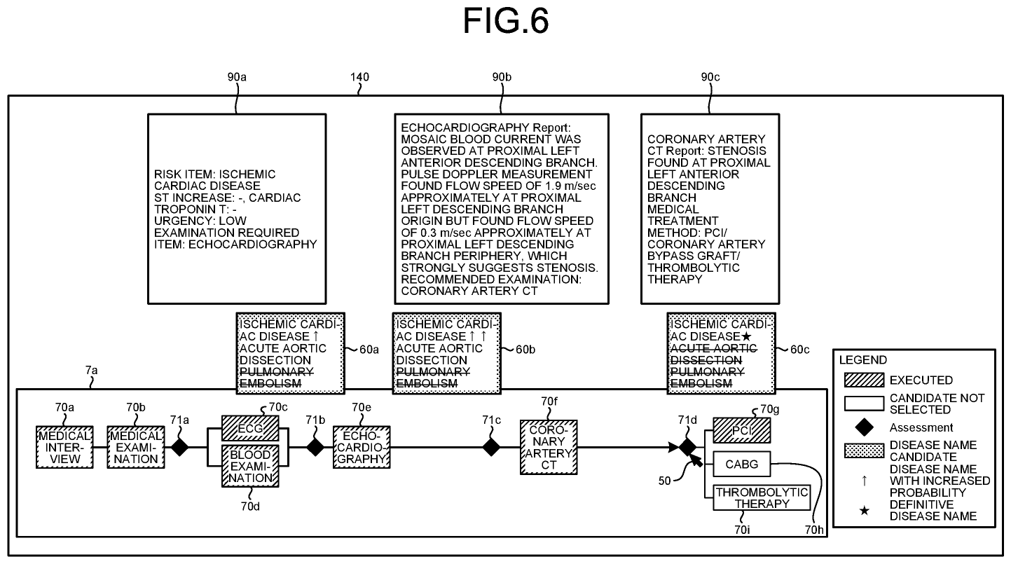

FIG. 6 is a diagram illustrating an exemplary browsing screen for the executed workflow 7a according to the first embodiment. The screen is used in a situation in which ex-post facto analysis or verification is performed for a medical action included in the workflow 7a. For example, the screen may be used when a doctor having executed a medical action included in the workflow 7a performs ex-post facto revision of own determination. The screen may be used for education of an unskilled doctor or for a conference at a medical institution, for example.

In the example illustrated in FIG. 6, a mouse pointer 50 operated by a user points to the bifurcation point 71d as an assessment for diagnosis name confirmation. In this case, the display control function 155 illustrates a part of the entire workflow 7a, for example, a part from the node 70a for a medical interview to an option for a medical action following the bifurcation point 71d.

The medical action following the bifurcation point 71d is PCI at a node 70g, CABG at a node 70h, or thrombolytic therapy at a node 70i. Among the three medical actions, PCI is selected by a doctor when the workflow 7a is executed. The display control function 155 causes the display device 140 to display the node 70g indicating PCI as a medical action selected by the doctor in a display aspect different from that of the node 70h indicating CABG and the node 70i indicating thrombolytic therapy as medical actions not selected by the doctor. For example, the display control function 155 may cause the display device 140 to display the medical action selected by the doctor in a color different from that of the medical actions not selected by the doctor.

In addition, the display control function 155 causes the display device 140 to display the information display fields 90a to 90c in which patient information and medical information corresponding to the nodes 70 and the bifurcation points 71 included in a displayed range in the workflow 7a are displayed, and the disease name candidate display fields 60a to 60c on the display device 140. In FIG. 6, the contents of the information display fields 90a to 90c in a situation in which ex-post facto analysis or verification is performed do not necessarily need to be identical to the contents of the information display fields 90a to 90c in a situation in which a medical action is executed by a doctor illustrated in FIG. 3. For example, information not displayed in a situation in which a medical action is executed by a doctor may be added to the information display fields 90 in a situation in which ex-post facto analysis or verification is performed.

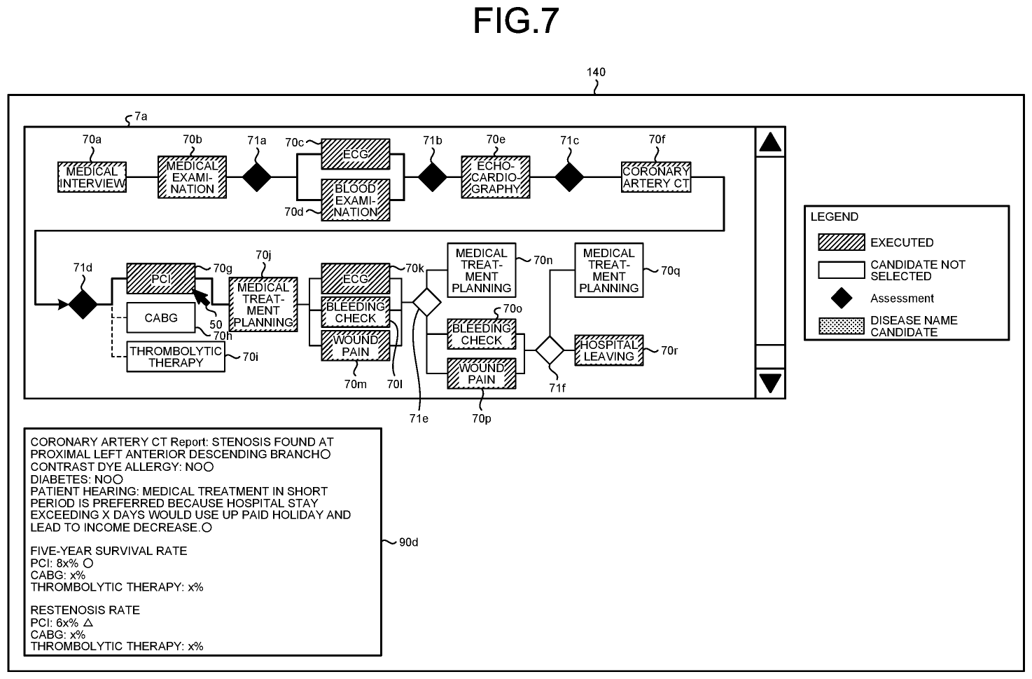

FIG. 7 is a diagram illustrating an exemplary continuation of the browsing screen for the workflow 7a in FIG. 6. In the example illustrated in FIG. 7, the mouse pointer 50 operated by the user points to the node 70g indicating “PCI” as the option selected by the doctor. In this case, the display control function 155 causes the display device 140 to display the option adopted by the doctor in association with any selection reason for the selection of the option. In the example illustrated in FIG. 7, when the user performs an operation to select the node 70g, the display control function 155 causes the display device 140 to display, near the node 70g, an information display field 90d in which any selection reason is displayed.

The display control function 155 causes the display device 140 to display, in the information display field 90d, any selection reason corresponding to the node 70g and searched from the reason record database of the memory 120. The selection reason is selected by the doctor from among the selection reason candidates 92a to 92c as described with reference to FIG. 5. In the example illustrated in FIG. 7, a result of a coronary artery CT report, allergy existence, pre-existing disease, past medical history, patient preference, and simulation results of the five-year survival rate and the restenosis rate for each option are displayed as selection reasons.

In FIG. 7, the display control function 155 causes the display device 140 to display, with a symbol, an estimation result by the estimation function 153 for whether each information included in the selection reasons corresponds to advantage or disadvantage for “PCI” selected by the doctor. For example, an item provided with a circle symbol among the selection reasons written in the information display field 90d corresponds to advantage for “PCI”. An item provided with a triangle symbol corresponds to disadvantage for “PCI”. Such a display aspect is exemplary and not restrictive.

When a medical action candidate selected by a doctor matches a medical action candidate recommended most by the estimation function 153, any recommendation reason generated by the estimation function 153 is displayed on the information display field 90d.

As illustrated in FIG. 2, the reception function 156 receives various operations by a user through the input interface circuit 130. For example, in a situation in which a medical action is executed by a doctor, the reception function 156 receives a selection of a candidate by the doctor from among a plurality of medical action candidates estimated by the estimation function 153. The reception function 156 also receives a selection of a selection reason candidate by the doctor.

In a situation in which ex-post facto analysis or verification is performed for an already executed medical action, the reception function 156 receives the designation of the workflow 7a as a browse target by a user. The reception function 156 also receives an operation by the user to designate a referral target among the nodes 70 or the bifurcation points 71 included in the designated workflow 7a.

The determination function 157 determines whether a medical action candidate selected by a doctor is different from a medical action candidate recommended most by the estimation function 153.

The collection function 158 collects patient information and medical information related to the workflow 7a including a plurality of medical actions. For example, in a situation in which ex-post facto analysis or verification is performed for an already executed medical action, the collection function 158 collects patient information and medical information associated with a medical action as a step included in the workflow 7a from the medical information storage apparatus 200, the nursing record storage apparatus 201, the electronic medical record system 202, the medical image storage apparatus 203, the statistics information storage apparatus 204, the ordering system 205, and the like.

Patient information associated with a medical action is, for example, patient information linked with identification information of a patient as a target of the medical action. The association of a medical action and patient information may be achieved, for example, by linkage of not only identification information of a patient but also date and time on which the medical action is performed or an order registered in the ordering system 205. Medical information associated with a medical action is, for example, a disease name of a patient as a target of the medical action, and statistics information and a medical care guideline related to an examination content included in the medical action.

The collection function 158 may specify related patient information and medical information, for example, based on the identification information of a patient as a target of a medical action and the date and time on which the medical action is performed. In the description of the present embodiment, collection of information to be displayed on the display device 140 and acquisition of information to be used for internal processing are separately performed by the collection function 158 and the acquisition function 151, respectively, but the acquisition function 151 may be integrated with the collection function 158.

The following describes the process of clinical decision support processing that is executed at the clinical decision support apparatus 100 configured as described above.

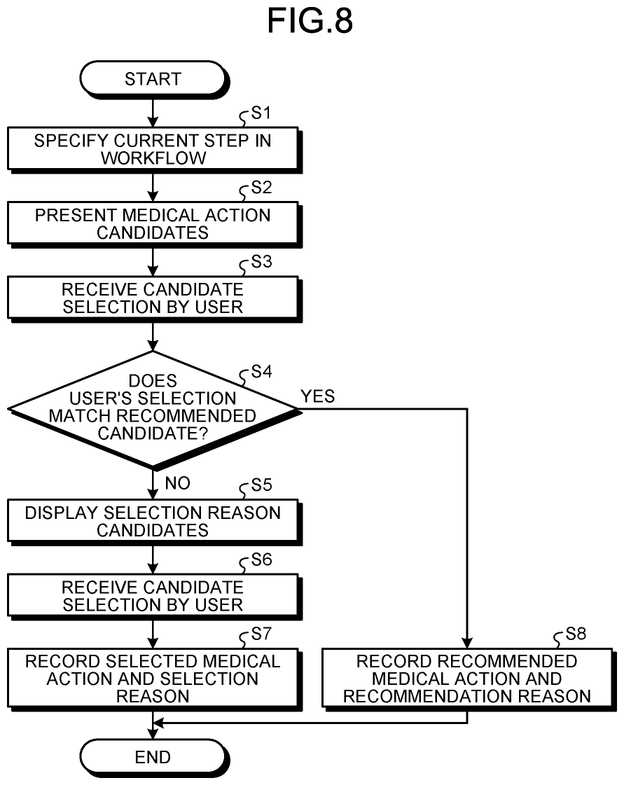

FIG. 8 is a flowchart illustrating an exemplary process of processing of supporting clinical decision according to the first embodiment; The flowchart is started when a doctor designates the target workflow 7a by, for example, designating identification information of a medical care target patient and the like.

The first specification function 152 specifies a current step among a plurality of medical actions included in the target workflow 7a (S1).

Subsequently, the estimation function 153 estimates any medical action candidate for an execution target following the current step. The display control function 155 presents the estimated medical action candidates to a doctor through causing the display device 140 to display the candidates (S2).

Then, the reception function 156 receives a selection of a medical action candidate by the user (S3).

Subsequently, the determination function 157 determines whether the medical action candidate selected by the user matches a medical action candidate recommended by the estimation function 153 (S4).

When the determination function 157 determines that the medical action candidate selected by the user does not match the medical action candidate recommended by the estimation function 153 (No at S4), the estimation function 153 estimates selection reason candidates 92. The display control function 155 causes the display device 140 to display the selection reason candidates 92 estimated by the estimation function 153 (S5).

Then, the reception function 156 receives a selection of a selection reason from among the selection reason candidates 92 by the user (S6).

The record processing function 154 records, in the memory 120, the medical action selected by the user from among the medical action candidates in association with the selection reason selected by the user from among the selection reason candidates 92 (S7).

When the determination function 157 determines that the medical action candidate selected by the user matches the medical action candidate recommended by the estimation function 153 (Yes at S4), the record processing function 154 records the medical action recommended by the estimation function 153 in association with a reason for the recommendation in the memory 120 (S8).

The following describes the process of processing that is executed at the clinical decision support apparatus 100 in a situation in which ex-post facto analysis or verification is performed for an already executed medical action.

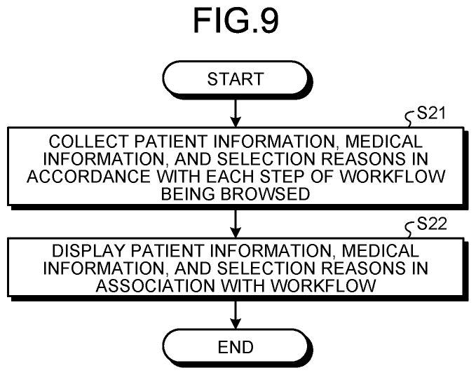

FIG. 9 is a flowchart illustrating an exemplary process of processing of supporting ex-post facto analysis or verification related to a medical action according to the first embodiment; The processing in the flowchart is executed, for example, when an operation to select the workflow 7a to be browsed by the user is received.

The collection function 158 collects patient information, medical information, and selection reasons in accordance with each step of the workflow 7a being browsed (S21).

The display control function 155 causes the display device 140 to display the collected patient information, the collected medical information, and selection reasons in association with the workflow 7a (S22).

In this manner, when collecting patient information and medical information related to the workflow 7a including a plurality of medical actions and displaying the workflow 7a on the display device 140, the clinical decision support apparatus 100 of the present embodiment causes the display device 140 to display the plurality of medical actions included in the workflow 7a, the collected patient information, and the collected medical information in time series and causes the display device 140 to display an option selected by a doctor in the workflow 7a in association with a reason for the selection of the option. Thus, the clinical decision support apparatus 100 of the present embodiment can facilitate ex-post facto analysis or verification of an already executed medical action.

When displaying the workflow 7a on the display device 140, the clinical decision support apparatus 100 of the present embodiment causes the display device 140 to display an option selected by a doctor and any other option not selected by the doctor in a distinguishable manner. Thus, the clinical decision support apparatus 100 of the present embodiment can further facilitate ex-post facto analysis or verification for the selection of a medical action by a doctor by displaying not only a medical action actually performed on a patient but also any other option not adopted.

When a doctor adopts an option different from an option recommended by the clinical decision support system, the clinical decision support apparatus 100 of the present embodiment stores the option adopted by the doctor in association with a reason for this selection in the memory 120, and causes the display device 140 to display the option adopted by the doctor in association with the selection reason at display of a record related to a past medical action on the display device 140. Thus, the clinical decision support apparatus 100 of the present embodiment can improve the accuracy of analysis related to medical action selection by a doctor.

For example, when a medical action for a patient is determined by a doctor and recording of any reason for the determination is insufficient although the determined medical action is recorded, it is difficult to perform ex-post facto verification or analysis of the validity of the determination in some cases. In particular, it is highly likely that there is some reason when an option other than an option recommended by the clinical decision support system is selected by a doctor. However, typically, the level of description in an electronic medical record or the like by a doctor is often left to discretion of the individual, and a reason for the clinical decision is not recorded in some cases. Instead, when the description of a reason for the clinical decision is required for a doctor who determines a medical action on a patient, disadvantage occurs in time and work needed for the description in some cases.

Since the clinical decision support apparatus 100 of the present embodiment records an option adopted by a doctor in association with a reason for the selection, the recorded information can be easily used as teacher data for machine learning or deep learning.

The clinical decision support apparatus 100 of the present embodiment estimates any candidate for a medical action to be executed next based on patient information and medical information of a medical action currently in progress, and causes the display device 140 to display the estimated medical action candidate as an option. Thus, the clinical decision support apparatus 100 of the present embodiment can support a clinical decision by a doctor in a situation in which a medical action is executed by the doctor.

When having estimated a plurality of medical action candidates, the clinical decision support apparatus 100 of the present embodiment determines a medical action candidate to be recommended most among the plurality of medical action candidates together with any reason for the recommendation, and estimates any reason candidate for the selection by the doctor when a medical action candidate selected by a doctor is different from the medical action candidate recommended most by the estimation function 153. In addition, the clinical decision support apparatus 100 of the present embodiment causes the display device 140 to display the estimated selection reason candidates and receives a selection from among the selection reason candidates by the doctor. Thus, with the clinical decision support apparatus 100 of the present embodiment, when an option other than the recommended option is selected by the doctor, it is possible to reduce a work load and a time for the doctor to write a reason and it is also possible to record a reason for the decision.

### Second Embodiment

In a second embodiment, the following describes the change of a medical treatment plan based on the difference between the medical treatment plan and an actual patient state.

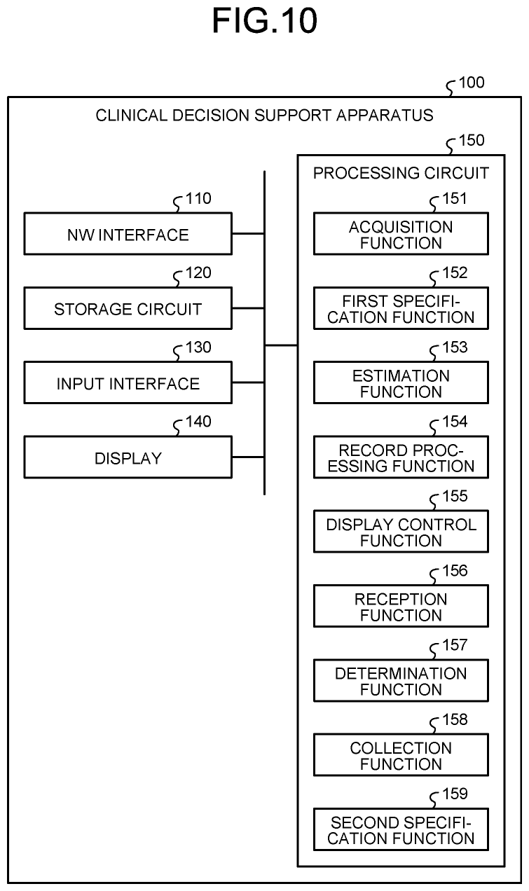

FIG. 10 is a diagram illustrating an exemplary configuration of the clinical decision support apparatus 100 according to the second embodiment. Similarly to the first embodiment, the clinical decision support apparatus 100 of the present embodiment includes the NW interface circuit 110, the memory 120, the input interface circuit 130, the display device 140, and the processing circuit 150.

The processing circuit 150 of the clinical decision support apparatus 100 of the present embodiment includes the acquisition function 151, the first specification function 152, the estimation function 153, the record processing function 154, the display control function 155, the reception function 156, the determination function 157, the collection function 158, and a second specification function 159. The acquisition function 151, the first specification function 152, the estimation function 153, the record processing function 154, the reception function 156, the determination function 157, and the collection function 158 have functions same as in the first embodiment.

The second specification function 159 specifies the difference between a state of a patient assumed in a medical treatment plan and the actual state of the patient included in patient information. The second specification function 159 determines the actual state of the patient based on, for example, any examination result and doctor's findings written in an electronic medical record.

The display control function 155 of the present embodiment has functions same as in the first embodiment and causes the display device 140 to display the difference specified by the second specification function 159 in association with a workflow.

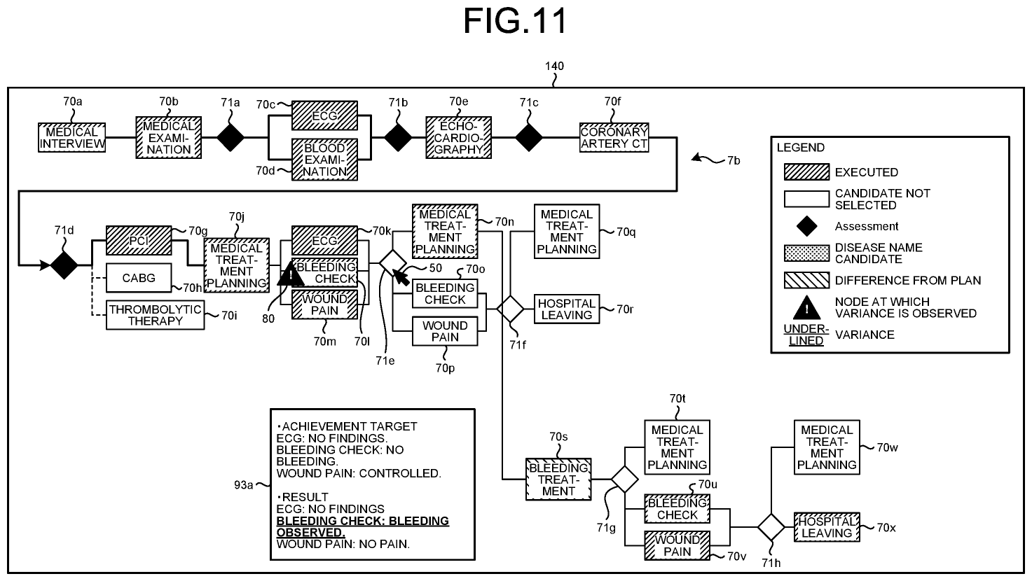

FIG. 11 is a diagram illustrating an exemplary browsing screen for an executed workflow 7b according to the second embodiment. In the example illustrated in FIG. 11, the mouse pointer 50 operated by a user points to a bifurcation point 71e. The bifurcation point 71e is an assessment for evaluating an assumed state of a patient, which is set at a node 70j for medical treatment planning, by comparison with patient information obtained through examinations corresponding to previously executed nodes 70k to 70m. In FIG. 11, the user performs ex-post facto referral of information compared and discussed through the assessment at the bifurcation point 71e.

In the example illustrated in FIG. 11, the patient state assumed at the node 70j for medical treatment planning is defined as an achievement target. For example, in FIG. 11, the state of “ECG: no findings. Bleeding check: no bleeding. Wound pain: controlled.” is an achievement target. The actual patient state is “ECG: no findings. Bleeding check: bleeding found. Wound pain: no pain.”

In the example illustrated in FIG. 11, bleeding is checked at the node 70l for bleeding check. However, a state with no bleeding is assumed at the node 70l for bleeding check in a medical treatment plan on which the workflow is based. In this case, the second specification function 159 specifies a result “bleeding check: bleeding found” as a difference between the patient state assumed in the medical treatment plan and the actual patient state. The difference is also referred to as “variance”. Variance indicates a state in which no outcome is achieved on a clinical path. The second specification function 159 specifies any node 70l at which variance occurs among a plurality of nodes 70 included in the workflow 7b.

In this case, the display control function 155 causes the display device 140 to display the difference specified by the second specification function 159 in association with the workflow 7b.

For example, when the bifurcation point 71e is selected by the user, the display control function 155 causes the display device 140 to display, near the bifurcation point 71e, patient information or medical information as a reason for a plan change at the bifurcation point 71e. In the example illustrated in FIG. 11, the display control function 155 causes the display device 140 to display, near the bifurcation point 71e, an information display field 93a in which the assumed patient state and the actual patient state are displayed. In addition, the display control function 155 emphatically causes the display device 140 to display, in the information display field 93a, the part “bleeding check: bleeding found” specified as the difference by the second specification function 159. The display control function 155 also causes the display device 140 to display a symbol 80 indicating an attention reminder in association with the node 70l at which variance occurs.

In the example illustrated in FIG. 11, it is assumed that the medical treatment plan is changed upon the occurrence of the difference. The display control function 155 causes the display device 140 to display, a node 70 for a medical action executed due to the change of the medical treatment plan and a node 70 for a medical action not executed due to the change of the medical treatment plan. Accordingly, both plans before and after the change are displayed on the display device 140.

When the medical treatment plan is changed due to a factor other than the occurrence of the difference, as well, the display control function 155 may cause the display device 140 to display information related to the factor. For example, the estimation function 153 may estimate whether to recommend a plan change at execution of a medical action by a doctor and may send a result of the estimation together with any reason to the display control function 155. In this case, the display control function 155 causes the display device 140 to display the contents and reason of the recommended plan change.

Before the plan is actually changed, the estimation function 153 may execute simulation of an outcome based on the changed plan. In this case, the display control function 155 may causes the display device 140 to display a result of the simulation in association with the workflow 7b.

In this manner, the clinical decision support apparatus 100 of the present embodiment specifies a difference between the patient state assumed in the medical treatment plan and the actual patient state included in patient information, and causes the display device 140 to display the specified difference in association with the workflow 7b. Thus, with the clinical decision support apparatus 100 of the present embodiment, any reason for a change of the medical treatment plan can be easily recognized by the user at ex-post facto analysis or verification of an executed medical action.

### Third Embodiment

In a third embodiment, any related part in a medical care guideline for illness suffered by a patient is displayed in workflow display.

Similarly to the first and second embodiments, the clinical decision support apparatus 100 of the present embodiment includes the NW interface circuit 110, the memory 120, the input interface circuit 130, the display device 140, and the processing circuit 150.

Similarly to the second embodiment, the processing circuit 150 of the clinical decision support apparatus 100 of the present embodiment includes the acquisition function 151, the first specification function 152, the estimation function 153, the record processing function 154, the display control function 155, the reception function 156, the determination function 157, the collection function 158, and the second specification function 159. The acquisition function 151, the first specification function 152, the estimation function 153, the record processing function 154, the reception function 156, the determination function 157, and the collection function 158 have functions same as in the first embodiment.

Medical information of the present embodiment includes medical care guidelines for various illnesses. Hereinafter, a medical care guideline is simply referred to as a guideline. As described in the first embodiment, medical information may include information other than a guideline.

The second specification function 159 of the present embodiment specifies a part related to patient information in a guideline included in medical information in a situation in which a medical action is executed by a doctor or a situation in which ex-post facto analysis or verification is performed for an already executed medical action.

In a particular method, the second specification function 159 may perform a search to determine whether an individual key word written in an electronic medical record is included in the guideline. The second specification function 159 may determine whether the guideline is infringed based on a combination of an individual key word written in an electronic medical record and a measured value included in an examination result. For example, when a key word “blood pressure” is described in a guideline for ischemic cardiac disease but a patient blood pressure “120/83 mmhg” written in an electronic medical record is defined as a normal value in the guideline, the second specification function 159 determines that the description is not related to the guideline. The second specification function 159 may search for a key word in a guideline written in, for example, a natural language.

The second specification function 159 may search the guideline based on a search condition set by a developer.

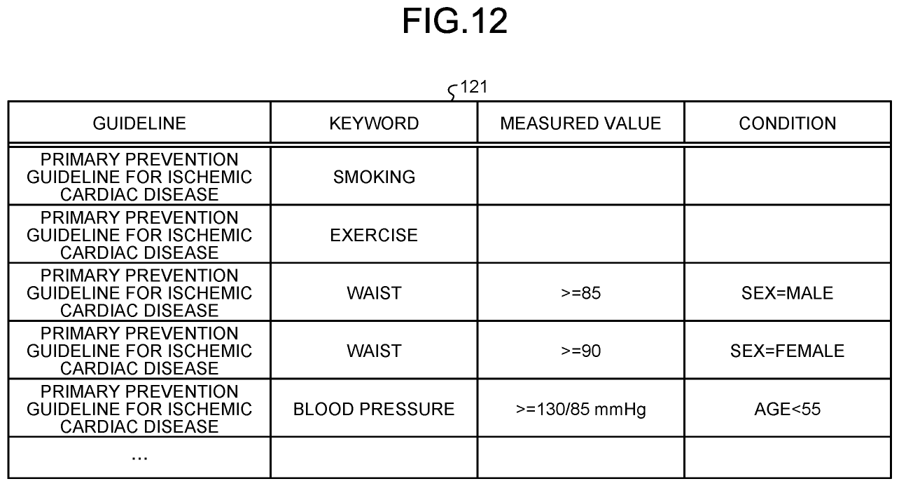

FIG. 12 is a diagram illustrating an exemplary search condition table 121 according to the third embodiment. The search condition table 121 defines a search condition for searching a guideline. For example, in the search condition table 121, a search target guideline, a search key word, a quantitative condition combined with the search key word, and a qualitative condition combined with the search key word are associated. The quantitative condition is, for example, a measured value in an examination.

The search target guideline does not necessarily need to be a guideline related to a medical treatment target illness in a workflow. For example, when a patient is in multi-morbidity, a guideline for any other illness in a medical treatment or a high-risk state as well as a guideline for a current medical treatment target illness may be a search target.

The search condition table 121 may be produced by a user instead of a developer. The second specification function 159 may produce the search condition table 121 by performing natural language analysis of an electronic medical record or the like. The search condition table 121 is an example of a search condition and not restrictive.

The display control function 155 of the present embodiment, which has functions same as in the first embodiment, causes the display device 140 to display a workflow related to a patient and a guideline related to an illness suffered by the patient.

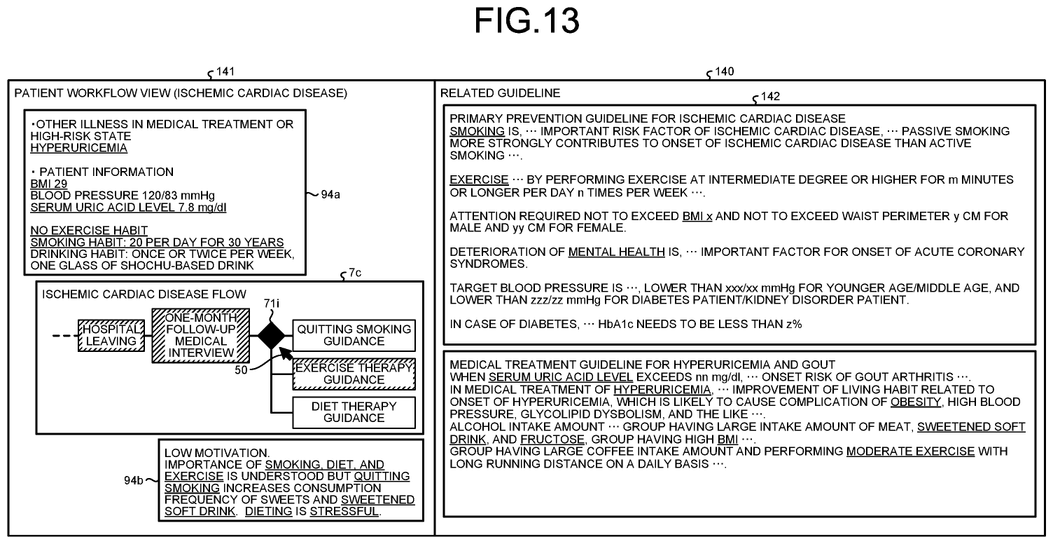

FIG. 13 is a diagram illustrating an exemplary browsing screen for an executed workflow 7c according to the third embodiment. As illustrated in FIG. 13, the display control function 155 causes the display device 140 to display, a first region 141 in which the workflow 7c and patient information are displayed and a second region 142 in which any related guideline is displayed.

In the example illustrated in FIG. 13, since a medical treatment target illness is ischemic cardiac disease, the display control function 155 causes the display device 140 to display a guideline related to ischemic cardiac disease on the second region 142. In addition, since the second specification function 159 specifies gout as a high-risk illness based on a patient state “hyperuricemia” written in an electronic medical record, the display control function 155 also causes the display device 140 to display a guideline for hyperuricemia and gout in the second region 142.

The display control function 155 emphatically causes the display device 140 to display any part related to the guidelines in medical information related to the workflow 7c other than the patient information and the guidelines. Hereinafter, in the present embodiment, medical information without qualification means medical information other than guidelines.

In the example illustrated in FIG. 13, the display control function 155 causes the display device 140 to display, in information display fields 94a and 94b, patient information related to a medical action included in the workflow 7c. In FIG. 13, since a bifurcation point 71i is selected by a user, the display control function 155 causes the display device 140 to display patient information related to an assessment of the bifurcation point 71i in the first region 141. In addition, the display control function 155 underlines any part with a description related to the guidelines in the patient information displayed in the information display fields 94a and 94b.

The display control function 155 also emphatically causes the display device 140 to display, with an underline or the like, any part related to patient information and medical information related to the workflow 7c in the description included in the guidelines. A method of emphatic display is not limited to underlining but may be emphatic display with text colors, markers, or the like.

When either medical action included in the workflow 7c is selected by the user, the display control function 155 emphatically causes the display device 140 to display any part related to the selected medical action in the guidelines, the patient information, or the medical information. For example, when a medical action is selected by the user in a case in which the workflow 7c includes options of a plurality of medical actions related to a medical treatment strategy, the display control function 155 further emphatically causes the display device 140 to display the description related to the selected medical action in the information display fields 94a and 94b and the guidelines.

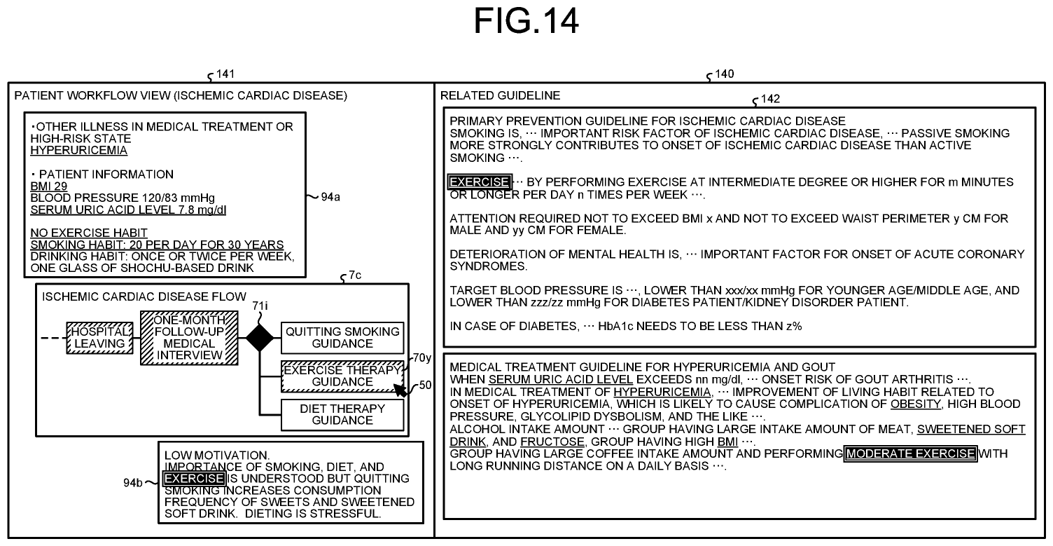

FIG. 14 is a diagram illustrating an exemplary emphatic display related to a selected medical treatment strategy according to the third embodiment; In FIG. 14, a node 70y corresponding to a medical action “exercise therapy guidance” is selected by the user. In this case, the display control function 155 further emphatically causes the display device 140 to display any part related to “exercise therapy guidance” among related parts that are emphatically displayed with underlines, which are described above with reference to FIG. 13. In FIG. 14, the display control function 155 performs emphatic display by surrounding with a frame but may perform emphatic display by highlighting, text flashing, or the like. When any part related to “exercise therapy guidance” in the guidelines and the information display field 94b is further emphatically displayed as illustrated in FIG. 14, the user can easily recognize that effects of exercise are written in both the guideline for ischemic cardiac disease and the guideline for hyperuricemia and gout and how the patient comments on exercise in a medical interview.

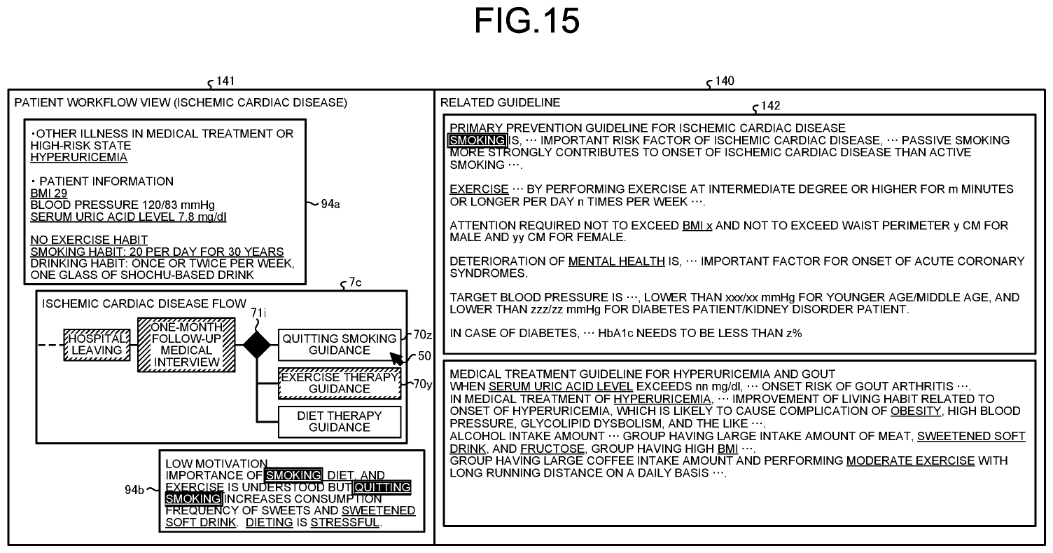

FIG. 15 is a diagram illustrating an exemplary emphatic display related to a medical treatment strategy different from that in FIG. 14. In FIG. 15, a node 70z corresponding to a medical action “quitting smoking guidance” is selected by the user. In this case, the display control function 155 emphatically causes the display device 140 to display any part related to “quitting smoking guidance”. In FIG. 15, a word “smoking” related to “quitting smoking” is emphatically displayed. With the emphatic display, the user can easily recognize that the guideline for hyperuricemia and gout has no description related to quitting smoking but the guideline for ischemic cardiac disease has such description. In addition, the user can easily recognize that negative information related to quitting smoking is included in a medical interview. Moreover, in the medical interview, it can be understood that the negative information related to quitting smoking gives negative influence on hyperuricemia. For example, it can be inferred, from contents written in the information display field 94b, that consumption of sweetened soft drink is increased due to quitting smoking, which adversely affects hyperuricemia.

In FIG. 15, among three options “quitting smoking guidance”, “exercise therapy guidance”, and “diet therapy guidance”, the options “quitting smoking guidance” and “diet therapy guidance” are not adopted but the option “exercise therapy guidance” is adopted. When the description related to each medical treatment strategy is checked in emphatic display as in FIGS. 14 and 15, the user can easily infer, at ex-post facto browsing of the workflow 7c, that exercise therapy, which has no negative influence but is effective for both diseases, is selected.

In this manner, the clinical decision support apparatus 100 of the present embodiment causes the display device 140 to display the workflow 7c related to a patient and a guideline related to an illness suffered by the patient. Thus, with the clinical decision support apparatus 100 of the present embodiment, even when the patient is in multi-morbidity, a determination can be easily performed with consideration on guidelines in a situation in which a medical action is performed by a doctor or a situation in which ex-post facto analysis or verification is performed for an already executed medical action.

Moreover, when either medical action included in the workflow 7c is selected by the user, the clinical decision support apparatus 100 of the present embodiment emphatically causes the display device 140 to display any part related to the selected medical action in guidelines, patient information, or medical information. With such emphatic display, the user can easily recognize related parts in the guidelines, and the patient information or the medical information.

### Fourth Embodiment

In a fourth embodiment, the following describes a case in which a target disease name candidate is changed halfway through a workflow.

Similarly to the first to second embodiments, the clinical decision support apparatus 100 of the present embodiment includes the NW interface circuit 110, the memory 120, the input interface circuit 130, the display device 140, and the processing circuit 150.

Similarly to the second and third embodiments, the processing circuit 150 of the clinical decision support apparatus 100 of the present embodiment includes the acquisition function 151, the first specification function 152, the estimation function 153, the record processing function 154, the display control function 155, the reception function 156, the determination function 157, the collection function 158, and the second specification function 159. The acquisition function 151, the first specification function 152, the estimation function 153, the record processing function 154, the reception function 156, the determination function 157, and the collection function 158 have functions same as in the first embodiment.

When a change of a candidate for the name of a disease selected as a medical action target is included in a workflow, the second specification function 159 of the present embodiment specifies information related to a disease name candidate after the change among patient information or medical information that corresponds to a medical action executed before the disease name candidate change. For example, the second specification function 159 may use the search condition table 121 described in the third embodiment or may specify any part related to a disease name candidate after the change by machine learning or deep learning.

The display control function 155 of the present embodiment causes the display device 140 to display the patient information or medical information specified by the second specification function 159.

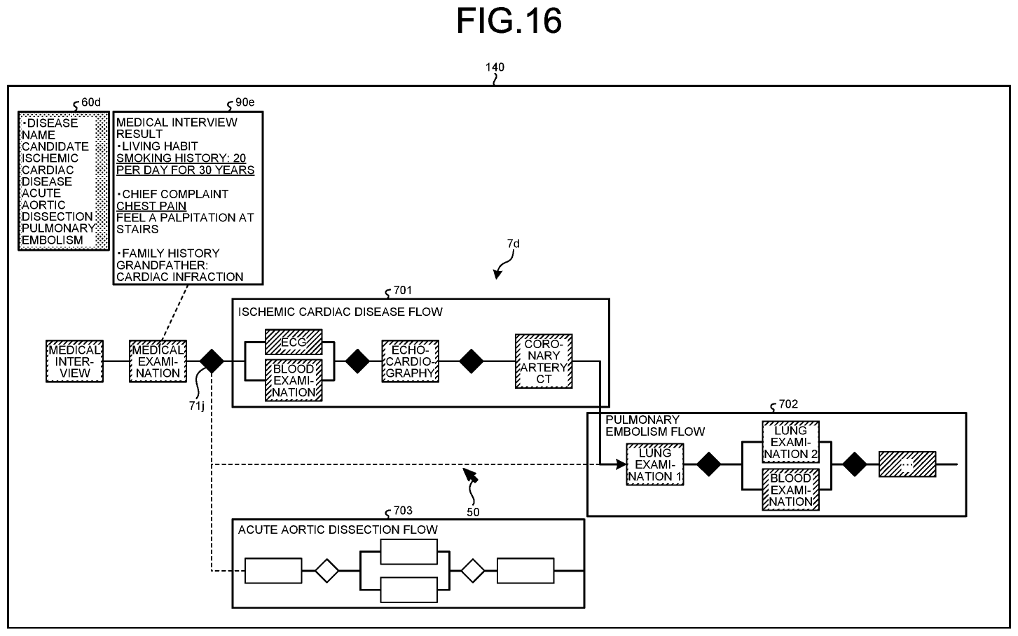

FIG. 16 is a diagram illustrating an exemplary browsing screen for an executed workflow 7d according to the fourth embodiment. FIG. 16 illustrates a case in which initial disease name candidates are three of “ischemic cardiac disease”, “acute aortic dissection”, and “pulmonary embolism” as described in a disease name candidate display field 60d. Thus, an ischemic cardiac disease flow 701, a pulmonary embolism flow 702, and an acute aortic dissection flow 703 corresponding to the three disease name candidates are displayed next to an assessment at a bifurcation point 71j. In the example illustrated in FIG. 16, “ischemic cardiac disease” among the disease name candidates is adopted as a disease name candidates with high probability or a disease name candidate with high risk. In this case, “ischemic cardiac disease” is a candidate for the name of a disease as a target of a medical action such as an examination. Thus, the ischemic cardiac disease flow 701 is executed as a step following the assessment at the bifurcation point 71j. As illustrated in FIG. 16, the bifurcation point 71j and the ischemic cardiac disease flow 701 are connected through a solid line in display. The ischemic cardiac disease flow 701 and the acute aortic dissection flow 703, which are not selected as a step executed after the bifurcation point 71j, are each connected to the bifurcation point 71j through a dashed line in display. The display aspect with solid and dashed lines illustrated in FIG. 16 is exemplary. The ischemic cardiac disease flow 701 includes any medical action for definitive diagnosis and medical treatment of “ischemic cardiac disease”.

However, consider a case in which a disease name candidate is changed to “pulmonary embolism”, which is not initially adopted, after the ischemic cardiac disease flow 701 is started. In this case, the ischemic cardiac disease flow 701 ends and the pulmonary embolism flow 702 starts as illustrated in FIG. 16. Such a flow change is performed, for example, when a doctor initially executes the ischemic cardiac disease flow 701 for a patient visiting a facility due to chest pain based on a chief complaint for a palpitation, family history, a risk of a fatal symptom, and the like, but the disease is pulmonary embolism, which causes the same symptom.

In this case, as illustrated in FIG. 16, the display control function 155 causes the display device 140 to display, with a dashed line or the like, a path from the bifurcation point 71j at which “pulmonary embolism” is not selected in reality to the pulmonary embolism flow 702. When the user selects the path from the bifurcation point 71j to the pulmonary embolism flow 702 with the mouse or the like, the display control function 155 emphatically causes the display device 140 to display patient information or medical information corresponding to a medical action related to a disease name candidate after the change, which is specified by the second specification function 159.

More specifically, the display control function 155 emphatically causes the display device 140 to display any part related to pulmonary embolism in patient information or medical information obtained before the pulmonary embolism flow 702 starts. In FIG. 16, the description “smoking history: 20 per day for 30 years” and “chest pain” are parts related to pulmonary embolism.

The parts related to a disease name candidate after the change in the patient information or medical information may be specified by the second specification function 159.

Moreover, the second specification function 159 of the present embodiment may specify, from among steps before the disease name candidate change, any step in which information related to a disease name candidate after the change is potentially obtained.

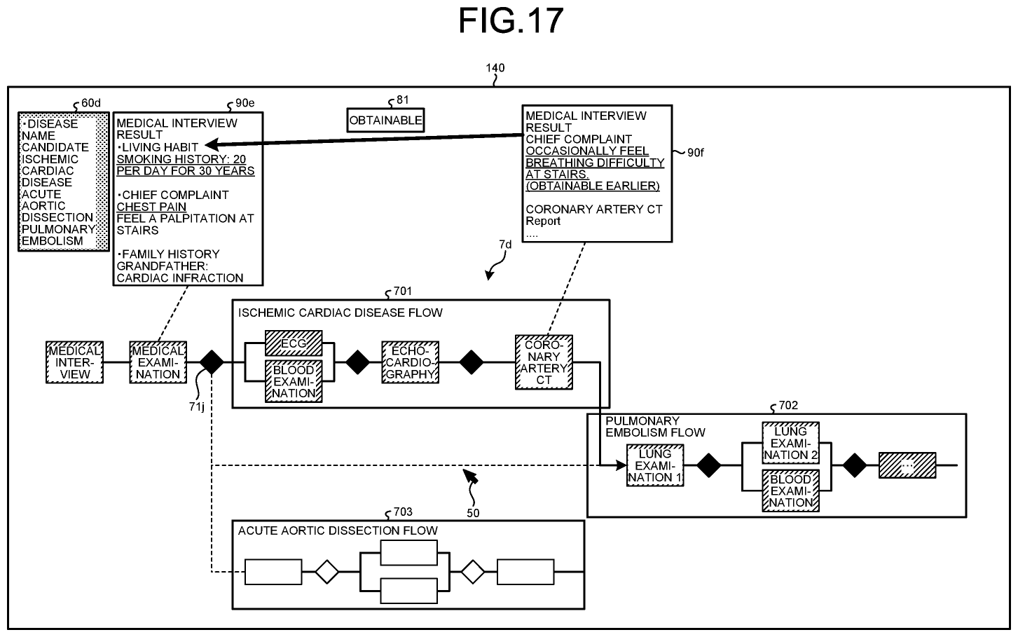

FIG. 17 is a diagram illustrating an exemplary information obtainable timing related to a past medical action according to the fourth embodiment; In the example illustrated in FIG. 17, the second specification function 159 specifies that information acquired in a medical interview at the last step of the ischemic cardiac disease flow 701 is information potentially obtained at the timing of an earlier medical interview.

In this case, the display control function 155 notifies the user that the information is information obtainable in the past by displaying a text message 81 “obtainable” and an arrow indicating the past step.

In the example illustrated in FIG. 17, the same name “medical interview” is used, but the second specification function 159 may determine obtainability depending on the relation between medical actions even when the medical actions do not have the completely same name. The second specification function 159 may indicate not only the existence of obtainability but also the degree of obtainability at stages. For example, when there are findings on a non-contrast CT report before a coronary arteriography CT report, the names of medical actions are not completely same but the medical actions are similar, and thus the second specification function 159 may determine, for example, “obtainability: “intermediate””.

In this manner, when a change of a candidate for the name of a disease selected as a medical action target is included in the workflow 7d, the clinical decision support apparatus 100 of the present embodiment specifies patient information or medical information that corresponds to a medical action executed before the disease name candidate change and is related to a disease name candidate after the change, and then causes the display device 140 to display the specified patient information or the specified medical information. Thus, the clinical decision support apparatus 100 of the present embodiment can facilitate a discussion of a medical action flow with which the disease name candidate after the change could be reached earlier.

### First Modification

In the first embodiment described above, screen display when the user performs an operation to select a medical action candidate and an operation to select a selection reason candidate in the workflow 7a in progress is described with reference to FIGS. 4 and 5. However, the screen display when the user performs the operation to select a medical action candidate and the operation to select a selection reason candidate is not limited to the above-described example.

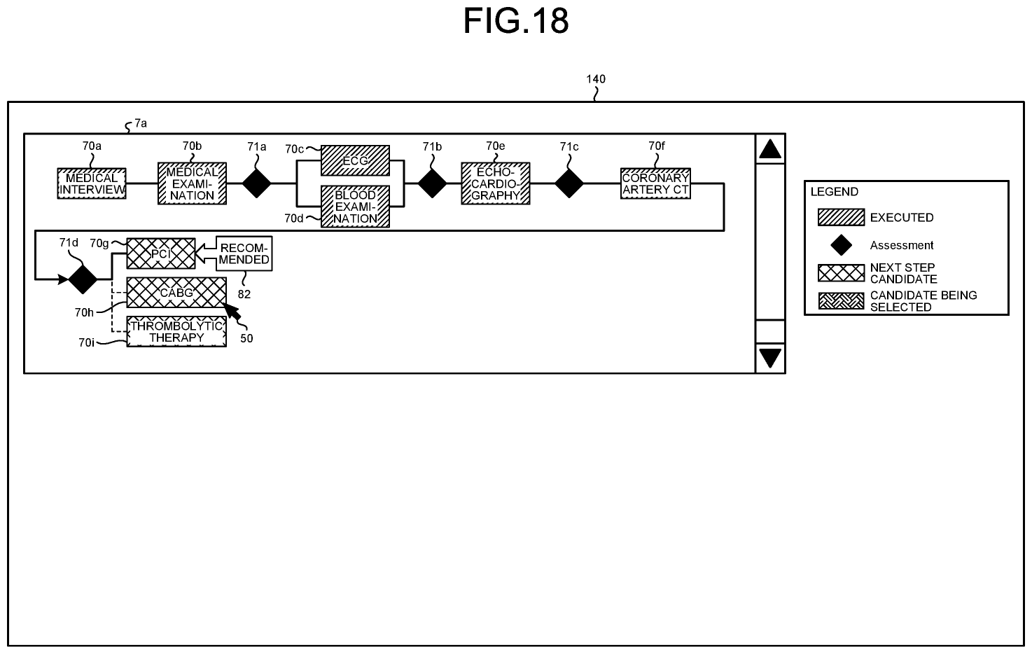

FIG. 18 is a diagram illustrating an exemplary selection screen for a medical action candidate according to a first modification; In the present modification, the display control function 155 causes the display device 140 to display, for a recommended medical action candidate, an image 82 indicating the recommendation in the workflow 7a in progress. In the selection screen illustrated in FIG. 18, PCI is displayed as a recommended option.

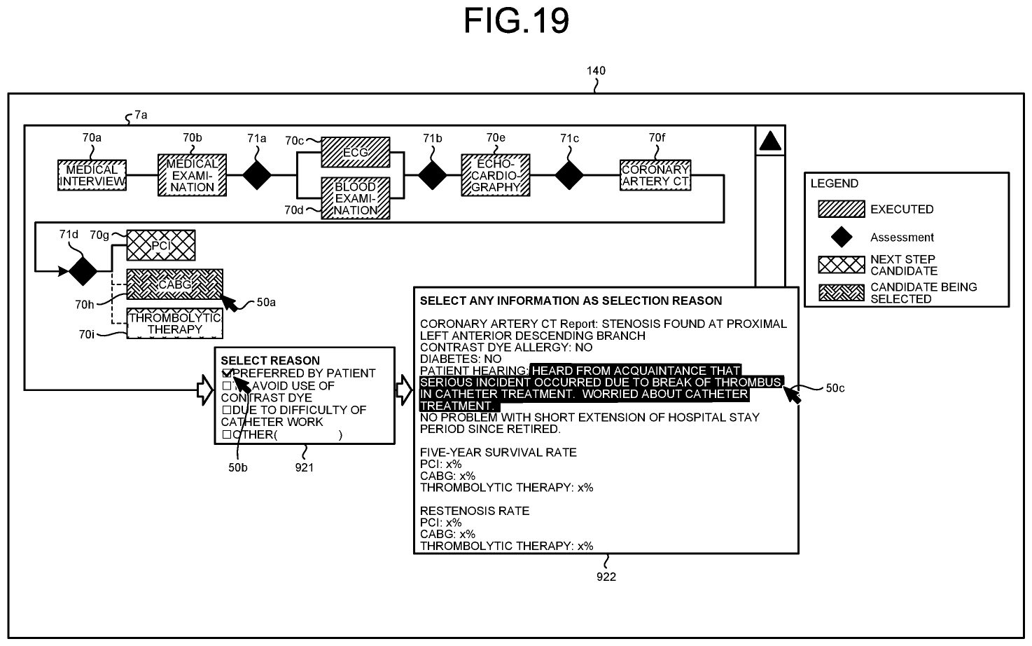

The following describes, with reference to FIG. 19, a case in which the user selects an option other than a recommended option on the selection screen illustrated in FIG. 18.

FIG. 19 is a diagram illustrating an exemplary selection screen for a selection reason candidate according to the first modification. In FIG. 19, a plurality of mouse pointers 50a to 50c are drawn on the display device 140 to describe operations by the user, but in reality, any one of them is displayed at once on the display device 140. Specifically, the pointer 50a, the pointer 50b, and the pointer 50c are displayed in the stated order in time series of operations by the user.

For example, the user selects CABG, which is not an option recommended by the system, on the selection screen illustrated in FIG. 18 described above. The user can select CABG by performing a click operation with the mouse pointer 50a placed on the node 70h indicating CABG.

In this case, the display control function 155 causes the display device 140 to display a selection reason candidate display field 921 on the workflow 7a in progress as illustrated in FIG. 19. A plurality of selection reason candidates with check boxes are displayed in the selection reason candidate display field 921. The plurality of selection reason candidates displayed in the selection reason candidate display field 921 may include information output as a reason for the estimation function 153 to recommend either medical action candidate. The display control function 155 may causes the display device 140 to display, as a selection reason candidate, information collected by the collection function 158 from the electronic medical record system 202, a guideline, or the like. Moreover, a plurality of selection reason candidates for a medical action may be registered in a database or the like in advance based on a guideline or the like. For example, the memory 120 may store any typical reason for the selection of PCI, CABG, or thrombolytic therapy by a doctor or the like in association with PCI, CABG, or thrombolytic therapy.

For example, the user can select a selection reason by performing a click operation with the mouse pointer 50b placed on a check box corresponding to either selection reason candidate. In FIG. 19, “patient preference” is selected as a selection reason. Means for the reception of selection by the user is not limited to a check box. In addition, a field in which the user can input a selection reason by text like an option “other” illustrated in FIG. 19 may be provided in the selection reason candidate display field 921.

After a selection reason for a medical action is selected in the selection reason candidate display field 921 by the user, the display control function 155 may also causes the display device 140 to display, on the workflow 7a in progress, a consult data candidate display field 922 in which the user can select consult data associated with the medical action and the reason. In the example illustrated in FIG. 19, with the mouse pointer 50c, the user selects, as consult data, information obtained from the patient in a medical interview, “Heard from an acquaintance that a serious incident occurred due to break of a thrombus in a catheter treatment. Worried about a catheter treatment.” The information selected by the user in the consult data candidate display field 922 is displayed in association with the medical action and the reason on the browsing screen for the executed workflow 7a.

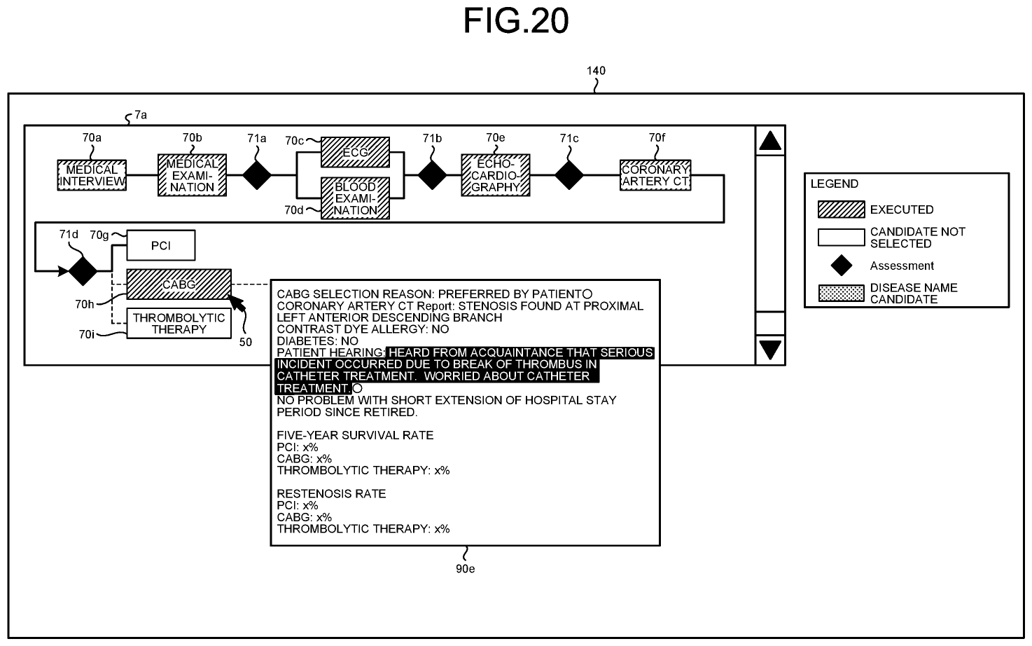

FIG. 20 is a diagram illustrating an exemplary browsing screen for the executed workflow 7a according to the first modification. As illustrated in FIG. 20, when the user places the mouse pointer 50 on, for example, the node 70h indicating CABG after the execution of the workflow 7a, the display control function 155 causes the display device 140 to display, in an information display field 90c, the CABG selection reason and consult data selected by the user in FIG. 19. The display control function 155 may causes the display device 140 to display, as consult data, information other than the information selected by the user. In this case, as illustrated in FIG. 20, the display control function 155 may emphatically cause the display device 140 to display the information selected by the user as compared to the other information.

### Second Modification

In each embodiment described above, the clinical decision support apparatus 100 has both the function to support decision when a doctor executes a medical action and recording of information related to the decision and the function to support ex-post facto analysis or verification of an already executed medical action, but any of these functions may be executed at a device different from the clinical decision support apparatus 100. For example, the clinical decision support apparatus 100 may include any one of the functions described with reference to the flowchart in FIG. 8 and the function described with reference to the flowchart in FIG. 9.

### Third Modification

Some or all of the functions of the clinical decision support apparatus 100 described above in each embodiment may be implemented in a cloud environment.

### Fourth Modification

The workflows 7a to 7d are exemplary medical records in the embodiments described above, but a medical record only needs to be a record in which a plurality of medical actions are associated in time series and may be a format other than the workflows 7a to 7d.

Various kinds of data described in the present specification are typically digital data.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising a processing circuitry configured to execute:
collecting patient information and medical information related to a medical record including a plurality of medical actions;
causing a display device to display the medical record by displaying the plurality of medical actions, the collected patient information, and the collected medical information in time series and displaying an option selected in the medical record by a doctor in association with any reason for the selection of the option; and
causing the display device to display the option selected by the doctor and any option not selected by the doctor in a distinguishable manner.

2. The medical information processing apparatus according to claim 1, wherein
when an option different from an option recommended by a clinical decision support system is adopted by a doctor, the processing circuitry stores the option adopted by the doctor in association with a reason for the selection in a memory, and
when displaying a record related to a past medical action on the display device, the processing circuitry causes the display device to display the option adopted by the doctor in association with the reason.

3. The medical information processing apparatus according to claim 1, wherein the processing circuitry
estimates a candidate for a medical action to be executed next based on patient information and medical information related to a medical action currently in progress, and
causes the display device to display the estimated medical action candidate as an option on the display device.

4. The medical information processing apparatus according to claim 3, wherein
when having estimated a plurality of medical action candidates, the processing circuitry determines a medical action candidate to be recommended most among the plurality of medical action candidates together with a reason for the recommendation,
the processing circuitry receives a selection of any of the plurality of medical action candidates by the doctor,
when the medical action candidate selected by the doctor is different from the estimated most recommended the medical action candidate, the processing circuitry estimates any reason candidate for the selection by the doctor,
the processing circuitry causes the display device to display the estimated selection reason candidates, and
the processing circuitry receives a selection from among the selection reason candidates by the doctor.

5. The medical information processing apparatus according to claim 3, wherein the processing circuitry
specifies a difference between a state of a patient assumed in a medical treatment plan and the actual state of the patient included in the patient information, and
causes the display device to display the specified difference in association with the medical record.

6. The medical information processing apparatus according to claim 1, wherein the processing circuitry causes the display device to display the medical record related to a patient and a guideline related to an illness suffered by the patient.

7. The medical information processing apparatus according to claim 6, wherein the processing circuitry emphatically causes the display device to display any part related to the guideline in the patient information and the medical information related to the medical record.

8. The medical information processing apparatus according to claim 6, wherein, when either medical action included in the medical record is selected by a user, the processing circuitry emphatically causes the display device to display any part related to the medical action in the guideline, the patient information, or the medical information.

9. The medical information processing apparatus according to claim 1, wherein
when a change of a disease name candidate selected as a medical action target is included in the medical record, the processing circuitry specifies the patient information or the medical information related to the disease name candidate after the change in the patient information or the medical information corresponding to a medical action executed before the change of the disease name candidate, and
the processing circuitry causes the display device to display the specified patient information or the specified medical information.
